(19) United States
(12) Patent Application Publication
(10) Pub. No.: US 2018/0010149 A1
(43) Pub. Date: Jan. 11, 2018

(54) PLASMONIC NANOCAVITY-BASED CELL THERAPY METHOD AND SYSTEM

(71) Applicant: PRESIDENT AND FELLOWS OF HARVARD COLLEGE, Cambridge, MA (US)

(72) Inventors: Eric Mazur, Cambridge, MA (US); Nabiha Saklayen, Cambridge, MA (US); Marinus Huber, Somerville, MA (US); Nicolas Vogel, Erlangen (DE); Marinna Madrid, Cambridge, MA (US)

(21) Appl. No.: 15/546,752

(22) PCT Filed: Feb. 5, 2016
(86) PCT No.: PCT/US16/16801

§ 371 Date: Jul. 27, 2017

Related U.S. Application Data
(60) Provisional application No. 62/113,115, filed on Feb. 6, 2015.

(51) Int. Cl. C12N 15/87; C12M 1/42

(52) U.S. Cl. CPC C12N 15/87; C12M 35/02

(57) ABSTRACT

In one aspect, a structure for use in transfecting cells is disclosed, which comprises a matrix supporting a plurality of cavities, each cavity having an opening characterized by a rim and an inner surface subtending and/or extending from said rim. An electrically conductive coating is disposed on a top surface of the substrate between, and connecting, the rims of the cavities. A layer of an electrically conductive material can also coat at least a portion of each cavity's inner surface. At least one dimension of each cavity is in a range of about 50 nm to about 3.5 microns, e.g., in a range of about 100 nm to about 1 micron, or in a range of about 200 nm to about 800 nm, or in a range about 200 nm to about 500 nm. In some cases, all dimensions of the cavity (e.g., X, Y, an Z-Cartesian dimensions) are in the aforementioned ranges.

### RELATED APPLICATION

The present application claims priority to provisional application No. 62/113,115 filed on Feb. 6, 2015 and entitled “Plasmonic Nanocavity-Based Cell Therapy Method and System,” which is herein incorporated by reference in its entirety.

### FEDERALLY SPONSORED RESEARCH

This invention was made with Government support under National Science Foundation (NSF) awards PHY-1219334 and PHY-1205465. The Government has certain rights in the invention.

### BACKGROUND

The present invention relates generally to systems and methods for causing plasmon-mediated poration of biological cells, e.g., by exciting localized plasmons in a metal coated matrix supporting a plurality of nanocavities.

There is currently a great interest in delivering a variety of agents into biological cells. For example, gene therapy, which is expected to play a major role in the coming years in treating a variety of illnesses from Alzheimer's disease to diabetes, generally relies on two categories of delivery methods: viral and non-viral. In viral methods, genetically engineered viruses target and deliver desired DNA material to a cell's nucleus. Even though such viral methods can achieve gene expression efficiently, their use has been limited in both clinical trials and laboratory assays due to a risk of inducing cellular-specific immune responses and fatal toxicity.

The non-viral methods can address many shortcomings of the viral methods. One such non-viral method, commonly known as opto-transfection, was introduced in early 1990's and has become popular for transfecting cells. In such a method, femtosecond radiation pulses can be focused onto the cell membrane using a high numerical aperture objective to form a spatially-confined spot of damage, or transient pore, through which DNA can diffuse into the cell.

Such conventional opto-transfection methods can have certain desirable features, such as low toxicity, relatively high efficiency, and spatial selectivity. But they also suffer from a number of shortcomings. For example, the use of these methods is limited to targeting and porating cells one at a time, which can impede their wide spread application.

Accordingly, there is a need for enhanced systems and methods for causing cell poration.

### SUMMARY

In one aspect, a structure (which is also herein referred to in many embodiments as a plasmonic structure) for use in transfecting cells is disclosed, which comprises a matrix supporting a plurality of cavities, each cavity having an opening characterized by a rim and an inner surface subtending and/or extending from said rim. An electrically conductive layer coats at least a portion of the matrix. For example, an electrically conductive coating can be disposed on a top surface of the matrix (substrate) between, and connecting, the rims of the cavities. A layer of an electrically conductive material can also coat at least a portion of each cavity's inner surface. In some embodiments, an entire inner surface of each cavity is coated with an electrically conductive material. In other embodiments, an electrically conductive layer coats a portion of an inner surface of each cavity, for example, in the form of a disk at the bottom of the cavity. Further, in some cases, an entire top surface of the matrix can be coated with an electrically conductive material. In some cases, some of the cavities can be at least partially coated with an electrically conductive material while other cavities are not coated. Moreover, in some cases, different electrically conductive materials can be used to coat at least a portion of a top surface of the matrix and at least a portion of an inner surface of one or more of the cavities. At least one dimension of each cavity is in a range of about 50 nm to about 3.5 microns, e.g., in a range of about 100 nm to about 1 micron, or in a range of about 200 nm to about 800 nm, or in a range about 200 nm to about 500 nm. In some cases, all dimensions of each of the cavities (e.g., X, Y, an Z-Cartesian dimensions) are in the aforementioned ranges.

In some embodiments, the conductive coating, e.g., a metal coating, of each cavity covers at least a portion of the cavity's inner surface at the bottom of the cavity. In some embodiments, the conductive coating of each cavity covers the entire inner surface of the cavity. Further, in some embodiments, the electrically conductive coating that covers the upper (top) surface of the matrix can be a contiguous surface extending between the openings of the cavities. By way of example, the conductive material that coats the top surface of the substrate can be the same conductive material that coats at least a portion of the cavity's inner surface. In some other embodiments, the conductive material forming the conductive coating on the top surface of the substrate can be different from the conductive material coating at least a portion of the cavity's inner surface. For example, in some embodiments, a coating of titanium and gold can be applied to the entire inner surface of each cavity, e.g., by tilting and rotating the sample during metal evaporation. In some embodiments, a contiguous metallic layer forms a metal coating on the top surface of the substrate as well as the inner surface of each cavity.

In some embodiments, the electrically conductive layer coating the inner surfaces of the cavities can have a thickness in a range of about 10 nm to about 100 nm, e.g., in a range of about 50 nm to about 100 nm. As noted above, such a conductive layer can also coat the rim and/or the top surface of the matrix. The electrically conductive coating can be formed of any suitable electrically conductive material. By way of example, in some embodiments, a metal, such as gold and/or silver, is used as the electrically conductive material. In some embodiments, the electrically conductive coating can include two or more metal layers. For example, the coating can include an underlying titanium layer (e.g., with a thickness of about 2 nm), and an upper gold layer (e.g., with a thickness of about 30 nm).

As noted above, at least one dimension of each cavity can be in a range of about 100 nm to about 2 microns, e.g., in a range of about 200 nm to about 1 micron. This dimension of the cavity can be, for example, the diameter of the openings of the cavities or the depth of the cavities or both.

In some embodiments, the plasmonic structure is formed of biocompatible materials. Some examples of suitable biocompatible materials include, without limitation, gold, silver, or titanium nitride.

The cavities can have a plurality of different shapes. By way of example, a cavity can have a truncated spherical inner surface.

The top surface of the substrate and the cavities are configured to allow placing a plurality of cells over the top surface such that each cell extends at least partially over an opening of at least one of the cavities. As discussed in more detail below, in some embodiments, in response to the irradiation of the structure by a plurality of laser pulses, the conductive coatings on the top surface of the matrix and/or on at least a portion of the inner surface of each cavity can generate surface plasmons, which can lead to the generation of ‘hot spots,” or areas of near-field enhancement, typically at the bottom and the rim of the cavities. Without being limited to any particular theory, these hot spots can in turn cause the generation of bubbles in a medium in which the cells applied to the top surface of the matrix are disposed, where the bubbles can mediate the generation of pores in the cells membranes. In some embodiments, the irradiation of the structure by a plurality of laser pulses can mediate the generation of pores in the cells’ membranes via mechanisms other than plasmon excitation, e.g., via Ohmic heating of the structure's metal-coated surfaces. Again without being limited to any particular theory, in some instances, depending on the laser parameters, the electric field of the applied laser pulses may generate surface plasmons, which can in turn generate a sufficiently enhanced near-field to lead to the generation of a plasma in the surrounding medium. The free electrons generated from the plasma may recombine with molecules in the surrounding medium, generating an increase in heat in the surrounding medium to cause the formation of bubbles. The bubbles can then mediate the formation of pores in the cells membranes. Again, without being limited to any particular theory, in other instances, depending on the laser parameters, the electric field of the applied laser pulses may generate surface plasmons, which decay into thermalized electrons and transfer heat to an electrically conductive coating of the plasmonic structure, which in turn transfers heat to the surrounding medium to cause the formation of bubbles. In such instances, the formation of bubbles is not mediated by and thus does not necessitate the generation of an enhanced near-field. In another instance, again without being limited to any particular theory, the electric field of the applied laser pulses may generate surface plasmons, which decay into thermalized electrons and transfer heat to the electrically conductive coating of the plasmonic structure, causing thermal expansion of the conductive coating, e.g., nanostructures of the conductive coating, and generating a photoacoustic wave in the surrounding medium that generates sufficient mechanical stress to porate the cells membranes, without necessitating the formation of bubbles. Therefore, while in many embodiments, the illumination of the metal-structured substrate by the laser pulses can generate surface plasmons, which can in turn lead to the formation of a photoacoustic wave or bubbles, which can cause an increase in the permeability of the cells membranes, the mechanisms by which poration of cells membranes can be achieved using plasmonic structures according to the present teachings can span a broad range and should not be limited to any of the particular theories discussed herein. In some embodiments, the applied laser pulses can have a short duration, e.g., a duration in a range of about 10 femtoseconds (fs) to about 100 nm, e.g., in a range of about 1 fs to about 10 ns, or in a range of about 100 fs to about 1 picosecond (ps), such as in a range of about 100 fs to about 500 fs. Further, in some embodiments, CW (continuous-wave) laser radiation can be used to excite surface plasmons. For example, CW lasers emitting light in the visible portion of the electromagnetic spectrum can be employed.

In some embodiments, the matrix of the plasmonic structure is substantially transparent to laser radiation suitable for interacting with the electrically conductive coating of the matrix so as to mediate the generation of pores in the cells' membranes. For example, the applied laser radiation can excite plasmon modes in any of the conductive coating on the top surface of the substrate and/or that disposed on inner surfaces of the cavities. In some such embodiments, the plasmonic structure can be irradiated with radiation from below the cavities, e.g., via a surface of the matrix opposed to its top surface, for example, so as to excite plasmon modes. In some other embodiments, the plasmonic structure can be irradiated from above the cavities.

In a related aspect, a method of causing poration of biological cells is disclosed, which comprises placing one or more cells over a top surface of a matrix supporting a plurality of cavities, where the matrix is at least partially coated with an electrically conductive material, e.g., at least a portion of the top surface of the matrix and/or an inner surface of one or more of the cavities is coated with an electrically conductive material. In some embodiments, each cell extends at least partially over an opening of at least one of the cavities, where each of the cavities comprises an inner surface extending from the cavity's opening. A layer of an electrically conductive material can cover at least a portion of the inner surface of one or more of the cavities. At least one dimension of each cavity is in a range of about 50 nm to about 2 microns, e.g., in a range of about 200 nm to about 1 micron, or in a range of about 200 nm to about 500 nm. In some cases, all dimensions of each of the cavities (e.g., X, Y, and Z-Cartesian coordinates) are within the aforementioned ranges. The method further comprises irradiating the matrix with a laser radiation (e.g., laser pulses) such that an interaction of the laser radiation with the electrically conductive material coating at least a surface portion of the matrix mediates the generation of one or more pores in the membrane(s) of the cells. In some embodiments, the laser radiation includes a plurality of laser pulses. In some such embodiments, the applied laser pulses can generate surface plasmons (e.g., localized surface plasmons) in electrically conductive layers of the cavities and/or the conductive coating of the matrix such that the surface plasmons dissipate energy to generate localized heat for mediating the generation of pores in membranes of one or more of said cells.

In some embodiments, the matrix is substantially transparent to the applied laser pulses and the radiation is applied to the matrix from below the cavities.

In some embodiments, the applied laser pulses have a central wavelength in a range of about 400 nm to about 2 microns.

In some embodiments, the applied laser pulses have a duration in a range of about 10 fs to about 100 ns, e.g., in a range of about 500 ns to about 10 ns, or in range of about 50 fs to about 1 ps, or in range of about 100 fs to about 500 fs. Further, the energy of the pulses, their spot size and their repetition rate can be selected to obtain poration of the cells membranes. For example, the fluence of the pulses can be selected so as to obtain a desired poration of the cell membranes. By way of example, and without any limitation, the pulse energy can be in range of about 1 nJ to about 500 μJ and the spot size can be, e.g., in a range of about 1 μm to about 10 cm, e.g., in a range of about 10 μm to about 1 mm, though other values can also be used. Further, a wide range of pulse repetition rates can be employed, e.g., from single pulses to about 80 MHz. By way of example, the fluence of the applied pulses can be in a range of about 1 mJ/cm² to about 100 mJ/cm². In some embodiments, the applied laser radiation is weakly focused so as to illuminate a plurality of cavities concurrently. This allows poration in the membranes of a plurality of cells concurrently. In some embodiments, continuous-wave (CW) radiation can be applied to a plasmonic structure according to the present teachings to mediate the generation of pores in the membranes of cells disposed on the plasmonic structure.

In some embodiments, the plasmonic structure for the generation of pores in cells can comprise vertically stacked layers of a plurality of interconnected cavities.

In some embodiments, the cells in which membrane pores are generated can be transfected with an agent, e.g., a viral agent or a DNA molecule. By way of example, the cells can be disposed in a medium in which an agent of interest is present. The medium including the cells can be applied to a top surface of a plasmonic structure according to the present teachings. The plasmonic structure can then be exposed to laser radiation (e.g. laser pulses) so as to generate pores in the cells membranes, e.g., in a manner discussed above. The agent then pass through such pores, e.g., via induced kinesis and/or thermal diffusion, and transfect the cells.

Further understanding of various aspects of the invention can be obtained by reference to the following detailed description in conjunction with the associated drawings, which are described briefly below.

### BRIEF DESCRIPTION OF THE DRAWINGS

FIGS. 1A and 1B schematically depict a plasmonic structure according to an embodiment of the present teachings,

FIG. 2 is a schematic cross-sectional view of one of the cavities depicted in the plasmonic structure shown in FIGS. 1A and 1B,

FIG. 3 is a flow chart depicting various steps of a method for fabricating a plasmonic structure according to the present teachings,

FIG. 4 is a flow chart depicting various steps of a method according to the present teachings for generating pores in the cells membranes,

FIG. 5 schematically depicts a cell disposed on a surface of a plasmonic structure according to the present teachings spanning a plurality of the openings of the cavities of the plasmonic structure,

FIGS. 6A and 6B show simulated electric field distribution generated in a cavity of a plasmonic structure according to the present teachings in response to illumination with laser pulses having a duration of 100 fs,

FIG. 7 shows a top-view SEM image of gold-covered nanocavities of a plasmonic structure according to the present teachings,

FIG. 8A shows HeLa cancer cells adhered to a plasmonic structure according to an embodiment of the present teachings,

FIG. 8B shows that the HeLa cells depicted in FIG. 8A were successfully porated by employing a method according to an embodiment of the present teachings,

FIG. 8C is a fluorescence image obtained from porated cells after their uptake of Calcein-AM, indicating viability of those cells,

FIGS. 9A and 9B show different views of a cavity of a plasmonic structure according to the present teachings employed in a theoretical simulation of “hot spots” generated as a result of the interaction of radiation with the metal-coated cavities of the structure,

FIG. 10 schematically shows an embodiment of a plasmonic structure according to the present teachings, which includes vertically stacked layers of a plurality of interconnected cavities,

FIG. 11A is a fluorescence image of HeLa cells that have uptaken Calcein upon illumination with 4-ns laser pulses at a repetition rate of 10 Hz with a central wavelength of 1064 nm,

FIG. 11B is a fluorescence image of HeLA cells that have uptaken Calcein-AM after treatment with radiation according to the present teachings, showing that approximately 99% of the radiation-treated cells remained viable,

FIG. 12A is a fluorescence image of HeLA cells that have uptaken dextran upon illumination with 11-ns laser pulses at a repetition rate of 50 Hz with a central wavelength of 1064 nm,

FIG. 12B is a fluorescence image of HeLA cells that have uptaken Calcein-AM after treatment with radiation according to the present teachings, indicating that approximately 81% of the radiation-treated cells remained viable,

FIG. 13A is a fluorescence image of HeLA cells that have uptaken Calcein upon illumination with 100 fs laser pulses applied at a repetition rate of 10 kHz with a central wavelength of 800 nm,

FIG. 13B is a fluorescence image of HeLA cells that have uptaken Calcein-AM after treatment with radiation according to the present teachings, indicating that approximately 92% of the radiation-treated cells remained viable,

FIG. 14A is a fluorescence image of HeLA cells that have uptaken Dextran upon illumination with 100 fs laser pulses applied at a repetition rate of 10 kHz with a central wavelength of 800 nm, and

FIG. 14B is a fluorescence image of HeLA cells that have uptaken Calcein-AM after treatment with radiation according to the present teachings, indicating that approximately 79% of the radiation-treated cells remained viable.

### DETAILED DESCRIPTION

The present invention relates generally to plasmonic structures that can be used to change permeability of cells membranes via irradiation, e.g., via nanosecond or femtosecond pulses. As discussed in more detail below, in many embodiments, such a structure according to the present teachings can be in the form of a matrix supporting a plurality of nanocavities, which are at least partially coated with an electrically conductive material, such as a metal. In some embodiments, the change in the permeability of the treated cells can allow introducing a variety of agents, such as viral or non-viral agents, into those cells. Various terms are used herein consistent with their ordinary meanings in the art. In the embodiments discussed below, various plasmonic structures and methods for generating pores in cells membranes are discussed. In some cases, the generation of one or more pores in a cell membrane refers to transiently increasing the permeability of the cell's membrane such that various agents (e.g. viral and/or non-viral) can pass through the cell's membrane. In some cases, the generation of one or more pores in a cell's membrane refers to permanently increasing the permeability of the cell's membrane. The term “surface plasmon” refers to oscillation of conduction electrons stimulated by incident light. A resonance condition may be established when the frequency of the incident photons matches the natural frequency of the oscillating electrons. The term “localized surface plasmon” refers to a surface plasmon that is primarily confined within a spatial extent, for example, within dimensions of about tens of nanometers. The term “about” is used herein to modify a numerical value denotes a variation of at most 5% about that numerical value.

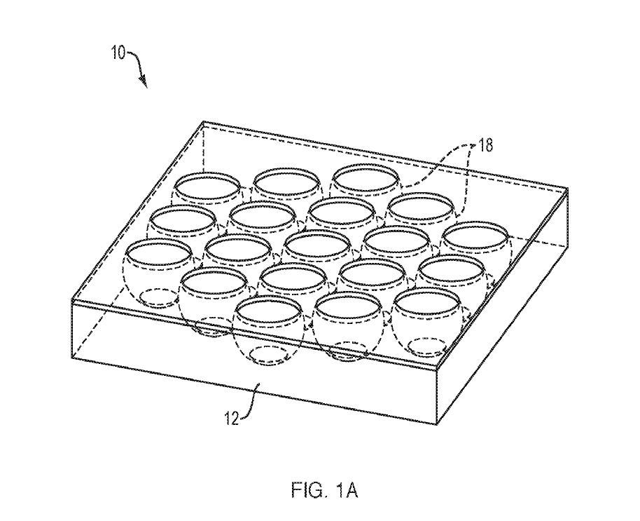

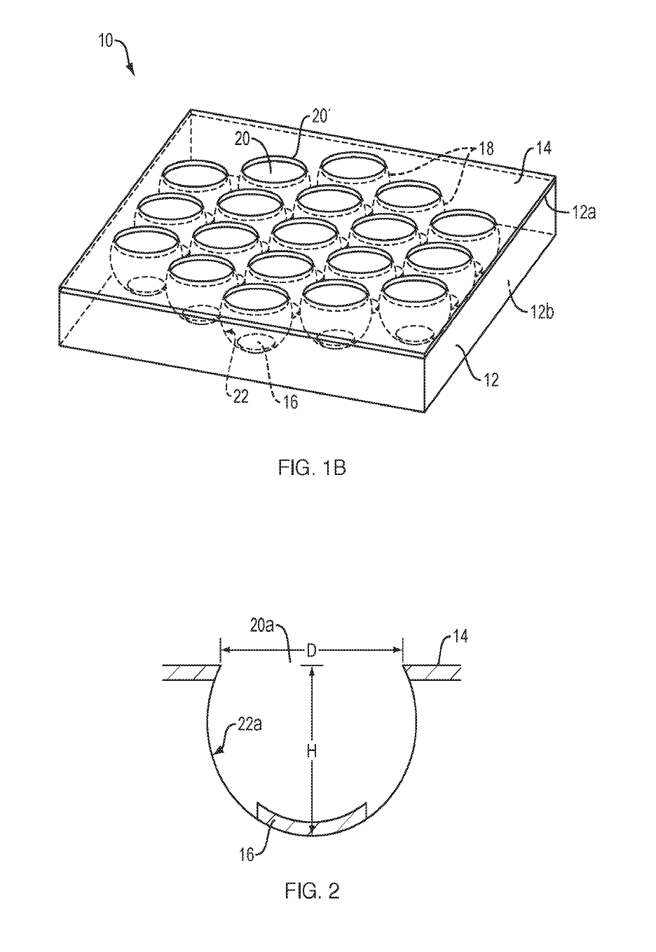

FIGS. 1A and 1B schematically depict a plasmonic structure 10 according to an embodiment of the present teachings. As discussed in more detail below, the plasmonic structure 10 can be used to cause poration of biological cells, for example, for transfecting those cells with viral and/or non-viral agents.

The plasmonic structure 10 includes a substrate 12 (herein also referred to as a matrix 12) having a top surface 12a and an opposed bottom surface 12b. A coating 14 of an electrically conductive material covers the top surface 12a. The coated top surface of the substrate is herein referred to as the top surface of the plasmonic structure. In this embodiment, the conductive material is gold, though other metals (or other electrically conductive materials) can also be used. In some embodiments, the substrate's top surface can be coated with TiN. The substrate 12 supports a plurality of cavities 18, which include openings (apertures) 20 defining rims 20′, each of which is surrounded by a portion of the top surface of the plasmonic structure 10. Each cavity includes an inner surface 22 that extends downward from the rim of its opening.

In this embodiment, a portion of the inner surface of each cavity is coated with an electrically conductive layer 14, which forms a disk-like metal surface at the bottom of the cavity. In other embodiments, an entire inner surface of each cavity can be coated with an electrically conductive layer. The electrically conductive layer can be formed of a variety of different materials. Some examples include, without limitation, metals, such as gold or silver, and TiN. In some other embodiments, the conductive layer 16 may form a contiguous metallic layer that overs the portions of the top surface of the substrate between the openings of the cavities as well as the inner surfaces of the cavities. In some other embodiments, the conductive layers covering the top surface of the substrate and those covering the inner surfaces of the cavities are the formed of different conductive materials.

The conductive coating 14 can be formed of any suitable electrically conductive material that can support, for example, excitation of a surface plasmon or provide plasmonic properties. In many embodiments, the conductive layer can be formed of a metal, such as, gold, silver, copper, titanium and/or chromium. In some embodiments, highly doped silicon can be used to form the conductive layers. In some embodiments, the conductive coating 14 can be formed of a stack of different metallic layers.

The thickness of the conductive layers 14/16 can be, for example, in a range of about 1 nm to about 100 nm.

In some embodiments, the cavities 18 have a sphere-like structure. For example, the inner surfaces of the cavities can be in the form of a truncated sphere. By way of example, FIG. 2 schematically depicts a cross-sectional view of one of the cavities 18 having an aperture 20a and an inner surface 22a that is in the form of a truncated sphere. The cavities 18 can have other shapes as well. In some embodiments, the cavities can have irregular shapes. As noted above, in this embodiment, a thin metal film is deposited at the bottom of each cavity.

In many embodiments, the plasmonic structure 10, or at least a portion thereof, such as the metal coating, is formed of a biocompatible material to minimize, and preferably prevent, any adverse interaction with biological cells disposed on the structure's top surface. Some examples of suitable biocompatible materials include, without limitation, gold and silver. In certain embodiments, the introduction of a functional group at the surface to prevent or tailor cell interaction can be achieved via linkage with a suitable anchor group. Some examples of suitable anchor groups include, without limitation, thiol functionalities, esters of phosphoric acids, carboxylic acids, dopamine or dopa-containing groups and peptide, and silanes. Such anchor groups can be linked to the metal-coated surfaces of the structure 10 using chemical processes known in the art.

In some embodiments, the substrate 12 can be substantially transparent to one or more electromagnetic radiation wavelengths to allow irradiating the substrate via a bottom surface thereof, i.e., from below the cavities, using one or more of those radiation wavelengths. The term “substantially transparent,” as used herein, means that the transmittance of the applied radiation through the substrate via the bottom surface thereof is sufficiently high to allow the interaction of the radiation with the electrically-conductive coating(s) of the substrate so as to mediate the generation of pores in the cells membranes, for example, by exciting the plasmonic modes of the cavities, via illuminating the structure from below the cavities.

With reference to FIG. 2, the cavities 18 have at least one dimension that is in a range of about 100 nm to about 2 microns, for example, in a range of about 100 nm to about 1 micrometer, or in a range of about 200 nm to about 500 nm. For example, the diameter (D) of the opening of the cavities can be in range of about 100 nm to about 2 microns, e.g., in a range of about 100 nm to about 1 micron or in a range of about 200 nm to about 500 nm, and the depth (H) of the cavities can be equal to or less than about 1 micron, e.g., in a range of about 100 nm to about 1 micron, or in a range of about 200 nm to about 500 nm. In some embodiments in which the cavity is substantially spherical, the depth of the cavity would correspond approximately to the diameter of the sphere.

In some embodiments, the substrate 12 comprises a cured sol-gel material, such as silicon dioxide, titania, alumina, zirconia, or any suitable polymeric material.

A nanoporous substrate according to the present teachings, such as the substrate 12, can be fabricated using a variety of different techniques, such as templating with a suitable porogen, soft lithography, or a variety of conventional microfabrication techniques, including electron beam lithography, deep UV photolithography or conventional photolithography. In certain embodiments, assemblies of colloidal particles can be used as a porogen to produce cavities in a continuous matrix. A porous matrix can then be generated by deposition of a continuous layer of a solid material onto the colloidal particles followed by removal of the templating particles. To achieve porosity, the particles can be selectively removed without removing the matrix material, for example by dissolution with a solvent or any other appropriate chemical component or via combustion at elevated temperature.

The colloidal particles can be assembled by various assembling methods, including but not limited to, spin coating, electrostatic deposition, electrophoretic deposition convective assembly, spray coating, assembly at an air/water or oil/water interface or doctor blading.

In some embodiments, polymeric colloidal particles can be used as porogens. Such colloidal particles can be synthesized by techniques known in the art, for example, via emulsion polymerization, surfactant-free emulsion polymerization, precipitation polymerization, suspension polymerization, and miniemulsion polymerization. (See, e.g., Vogel, N., Weiss, C. K., & Landfester, K. (2012) entitled “From soft to hard: the generation of functional and complex colloidal monolayers for nanolithography,”Soft Matter, 8(15), 4044-4061 which is herein incorporated by reference in its entirety, and in particular its pages 4047 and 4048.)

In some embodiments, the methods disclosed in Vogel et al. “Transparency and damage tolerance of patternable omniphobic lubricated surfaces based on inverse colloidal monolayers” published in Nature Communications (Jul. 31, 2013), which is herein incorporated by reference in its entirety, can be employed for forming a matrix supporting a plurality of nanocavities. This reference discloses crystallizing colloidal monolayers on the air-water interface of a crystallization dish by spreading a 1:1 water/ethanol colloidal dispersion onto the interface via a glass slide until the surface is completely covered. This can be followed by inserting the substrate into the subphase and depositing manually the monolayer by fishing out the slide. After drying, a closed packed monolayer uniformly can cover the substrate. For example, the colloids can be prepared by surfactant-free emulsion polymerization of styrene with acrylic acid as comonomer. An inverse monolayer can then be formed as follows. A solution of tetraethylorthosilicate (TEOS), HC1 (0.1 mol l 1) and ethanol with weight ratios of 1:1:1.5 can be prepared and stirred for 1 h. Then, it can be diluted with ethanol and spin-coated onto the monolayer-covered substrate (3,000 r.p.m., 30 s, acceleration 500). The colloids can be removed by combustion at 500 C (ramped from room temperature to 500 C for 5 h, 2 h at 500 C).

Vogel, N., Retsch, M., Fustin, C. A., del Campo, A. & Jonas, U. (2105) “Advances in colloidal assembly: The design of structure and hierarchy in two and three dimensions,” published in Chemical Reviews, 115(13), 6265-6311, which is herein incorporated by reference in its entirety, discloses methods for assembling polymeric particles, such as polystyrene and polymethylmethacrylate particles, with and without additional additives and crosslinkers, other particles formed from polymers, inorganic oxide particles, such as silica, titania, zirconia, alumina, iron oxide and other main group or transition metal oxides and mixtures thereof, and metallic nanoparticles, such as gold, silver, copper and titanium nitride. The particles can range in size from about 100 nm to several microns, e.g., 3 microns. Such particles can be used to form nanopores in a variety of different matrices, which can be formed of polymeric, oxidic or metallic materials. In some cases, the matrix can include nanoparticles fused together via attractive interactions or sintering. In some cases, the matrix can be formed via physical deposition techniques such as atomic layer deposition, chemical vapor deposition, sputter coating, thermal evaporation, sol-gel techniques, spin coating, spray coating or doctor blading, among other fabrication techniques. By way of example, the matrix can be deposited in layers having a thickness in a range of about 50 nm to a few microns, e.g., 3 microns, on an underlying substrate. By way of example, in some cases, the matrix can be a tetraethyl; orthosilicate layer, e.g., 750 nm high, with nanocavities generated by 1 micron-diameter polystyrene particles serving as the porogen. More generally, any combination of matrix material and templating porogen can be used as long as the porogen can be selectively removed from the matrix to form the porous structure.

The matrix can be formed on any substrate that is suitable for deposition of porogen particles, the matrix and selective removal of the porogen. Some examples of suitable substrates include, without limitation, glass , silicon wafer, metal, metal coated glass, polymeric materials, such as polystyrene, polymethylmethacrylate, polyetheretherketons, polyamides, polyesters, polysulfones, and other acrylic polymers.

As noted above and discussed in more detail below, in many embodiments, an electrically conductive layer can be deposited on at least a portion of a plurality of nanocavities supported by a matrix. In some cases, the conductive material can also coat at least a portion of the top surface of the matrix, and particularly around the perimeter of the openings of the nanocavities. Such a conductive layer can be formed, for example, via thermal evaporation, electron beam evaporation, atomic layer deposition, sputter coating or other deposition techniques. The conductive layer can be formed of a metal, such as gold, silver, copper, chromium, and titanium, titanium nitride, among others. In some embodiments, the metal coating can have a thickness in a range of about 10 nm to about 100 nm. In some cases, the metallic coating can be formed of two layers of metal. By way of example, in some embodiments, the metallic coating can include an underlying titanium layer (e.g., 2 nm thick) and an upper gold layer (e.g., 50 nm thick).

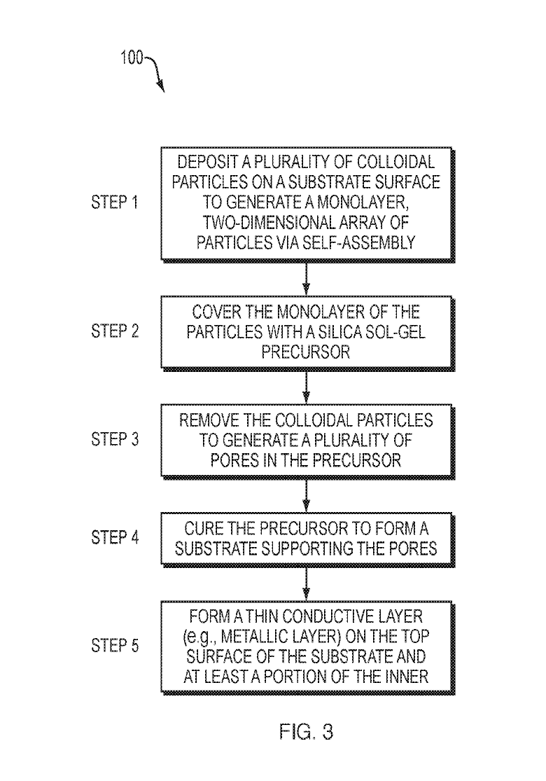

By way of example, in one method of fabricating the plasmonic structure 10, a colloidal self-assembly method can be employed. For example, with reference to flow chart 100 of FIG. 3, a plurality of colloidal particles can be deposited on a substrate (step 1) to generate a monolayer, two-dimensional array of the particles via a self-assembly process. Subsequently, a sol-gel precursor, e.g., a silica sol-gel, can be used to cover the particle monolayer, e.g., to a height of at least half the diameter (step 2). Generally, any suitable sol-gel precursor can be used. In some cases, the sol-gel precursor and the colloidal particles can be selected such that they are not soluble in the same solvent to allow removal of the colloidal particles in subsequent steps. Some examples of suitable sol-gel precursor materials include, without limitation, silicon dioxide, titania, alumina, and zirconia.

The colloidal particles can be subsequently removed (step 3) and the precursor can be cured (e.g. via heat treatment) (step 4) to form a substrate containing a plurality of pores (cavities).

A thin layer of an electrically conductive material, e.g., a thin layer of a metal, can then be formed on the top surface of the substrate and on at least a portion of the inner surfaces of the pores (step 5). By way of example, a thin metal film, e.g., a gold film, can be evaporated onto the top substrate surface including the pores to create a gold film surface on the top surface of the substrate and the bottom of each pore. This fabrication process is highly parallel, fast and inexpensive as it does not rely on expensive nanofabrication tools.

As noted above, a plasmonic structure according to the present teachings can be employed for generating pores in membranes of biological cells, which in turn allows transfecting cells with agents of interest. Some examples of such agents include, without limitation, plasmids, siRNA, mRNA. In addition to transfecting the cells with genetic material, the pores in the cell membranes can be employed to deliver other molecules, such as, dyes, drugs, and therapeutic agents, into the cells. In some embodiments, the molecules delivered to the cells can have a molecular weight in a range of about 500 Da to 2,000,000 Da.

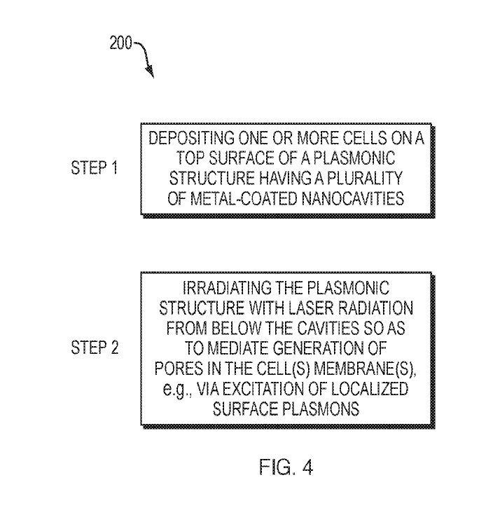

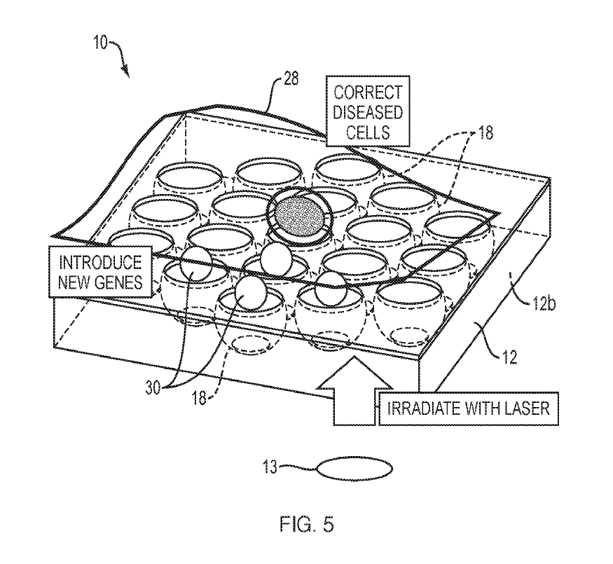

With reference to flow chart 200 of FIG. 4 and FIG. 5, in one method of causing cell poration according to the present teachings, one or more cells 28 can be disposed on a top surface of the plasmonic structure 10 over the cavities 18 (FIG. 4, step 1). The cells can be in a medium in which agents of interest 30, e.g., DNA molecules, are present. The plasmonic structure can then be irradiated from below the cavities (FIG. 4, step 2), i.e., via the bottom surface 12b of the substrate 12, with laser radiation pulses to which the substrate is substantially transparent such that the interaction of the radiation with the structure's electrically conductive coating can mediate the generation of pores in at least some of the cells 28. By way of example, the interaction of the laser pulses with the metal coating of the structure (e.g., the top coating and/or metal coating within the cavities) can excite localized surface plasmons, for example, at the interface of the cell medium and the metal coating. The localized surface plasmons can mediate the generation of pores in the cells' membranes, e.g., via heat generation that can result in generation of bubbles in a medium in which the cells are disposed. In some embodiments, the cells can be within a medium in which one or more agents of interest are present, which can diffuse through the pores generated in the cells membranes into the cells. As noted above, the cells can be transfected with a variety of different biological agents in this manner. Some examples of biological agents include, without limitation, DNA and RNA molecules and/or fragments, DNA-encoding plasmids, proteins, enzymes, nucleases such as Cas9, antibodies, viruses, dyes, among others.

In some embodiments, at least about 40%, or at least about 50%, or at least about 60%, or at least about 70%, or at least about 80%, or at least about 90%, or at least about 95%, or at least about 99%, and in some embodiments 100%, of the treated cells, i.e., the cells exhibiting a change in their permeability, remain viable after the treatment (i.e., after radiation treatment via application of laser pulses to a plasmonic structure according to the present teachings on which the cells are placed). The viability of the treated cells herein is determined based on their ability to show an intracellular enzymatic activity that occurs in untreated healthy cells. For example, the treated cells can be exposed to Calcein-AM, which is membrane permeable and hence is uptaken by all cells. Calcein-AM is not, however, naturally fluorescent. It fluoresces only when its ester bond is broken by an enzyme within the cell. Thus, if a treated cell that has uptaken Calcein-AM exhibits a fluorescent signal associated with Calcein-AM, then one can conclude that it has maintained its healthy enzymatic activity and will be herein considered as a viable cell.

In some embodiments, one or more optics 13, such as one or more lenses, can be employed to direct the radiation onto the plasmonic structure. In some embodiments, the optics are configured such that a plurality of cells are concurrently illuminated by the applied radiation. For example, the lenses can focus radiation onto the plasmonic structure at a numerical aperture in a range of about 0.025 to about 1.

In some embodiments, the applied laser pulses have a pulse duration in a range of about 50 fs to about 100 nanoseconds (ns), e.g., in a range of about 100 fs to about 1 picosecond, or a range of about 100 fs to 500 fs. In some embodiments, the pulse duration of the applied laser pulses can be in a range of about 1 ns to about 100 ns, e.g., in a range of about 4 ns to about 50 ns, or in a range of about 10 ns to about 30 ns, or in a range of about 10 ns to about 20 ns. The central wavelength of the pulses can be, for example, in a range of about 400 to about 2000 nm. In some embodiments, the applied pulses can have a fluence in a range of about 1 mJ/cm² to about 100 mJ/cm², e.g., in a range of about 10 mJ/cm² to about 80 mJ/cm², or in a range of about 20 mJ/cm² to about 50 mJ/cm². A variety of pulse repetition rates can be employed. By way of example, the repetition rate can range from a single pulse to a repetition rate of 80 MHz, e.g., from about 10 Hz to about 1 MHz.

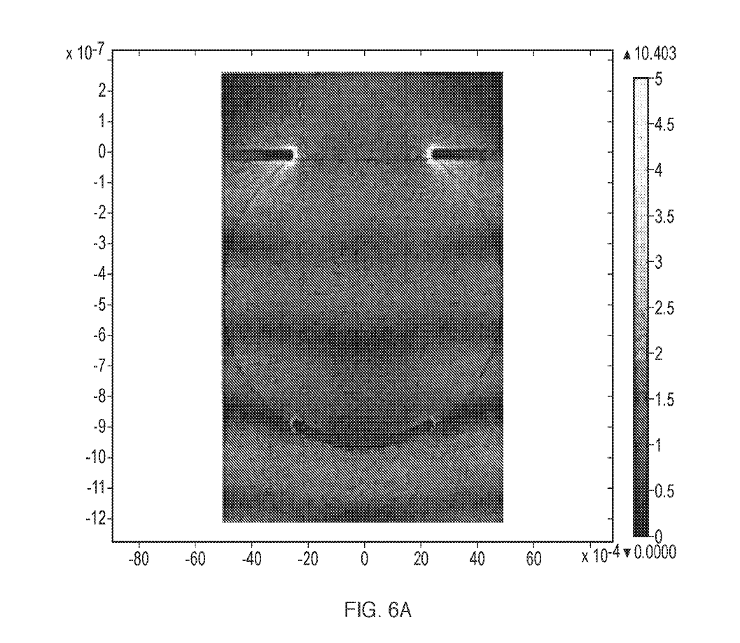

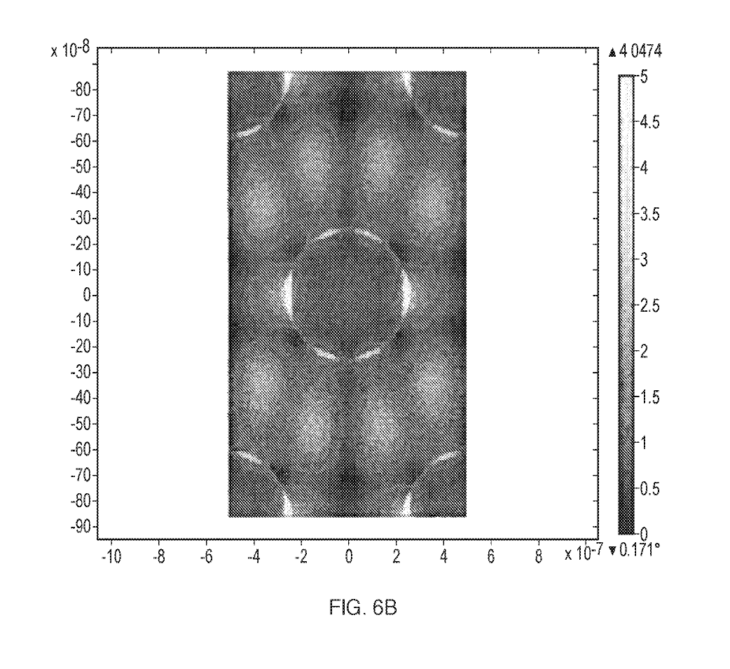

Simulation results show that in some embodiments the illumination of a structure according to the present teachings for generating pores in cells membranes can result in a strong near-field enhancement at the rim and the base of the cavities, which can decay over tens of nanometers. By way of example, FIGS. 6A and 6B show simulated electric field distribution generated in a cavity in response to illumination with laser pulses having a duration of 100 fs (the other parameters used in the simulation are discussed below in Example 2), resulting in top and bottom hotspot formation. Using a weakly focused laser beam, multiple cavities can be illuminated concurrently, leading to the formation of a plurality of hotspots across those cavities.

Without being limited to any particular theory, such localized surface plasmons, herein also referred to as “hot spots,” can cause localized heating via dissipation of the applied electromagnetic energy, which can in turn lead to generation of bubbles in the medium containing the cells at the proximity of the rims of the cavities. The contact of the bubbles with the cell membrane can form transient pores therein. The generated pores can in turn allow the delivery of agents of interest, e.g., DNA molecules, into cells, such as cancer cells for gene therapy. Again without being limited to any particular theory, in some embodiments, the applied pulses can effect the generation of photoacoustic waves, which can in turn mediate a change in the permeability of the cells' membranes, e.g., via formation of transient or permanent pores in the cells' membranes.

One advantage of the above method is that it allows targeting multiple cells disposed on the top surface of the plasmonic structure 10 at once by illuminating the structure with a weakly focused laser light. By way of example, the numerical aperture (NA) of the radiation illuminating the plasmonic structure can be in a range of about 0.025 to about 1. In response, the plasmonic structure focuses the laser light to multiple spots on the metal-covered surface to generate, at least in some embodiments, bubbles that can cause poration of the membranes of the cells.

As noted above, once the cells are porated, a variety of agents can be introduced into the cells via such pores.

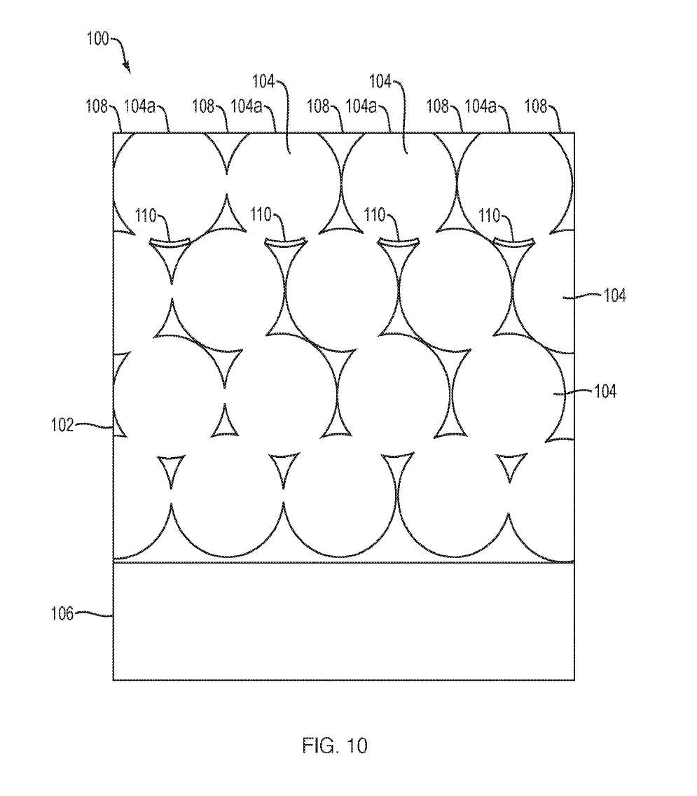

With reference to FIG. 10, in some embodiments, rather than using one layer of cavities, a vertically stacked layers of a plurality of interconnected cavities is employed to form a plasmonic structure according to some embodiments of the present teachings. For example, FIG. 10 shows a plasmonic structure 100 according to an embodiment, which includes a substrate (herein also referred to as a matrix) 102 that supports a plurality of cavities 104. In this embodiment, the supporting substrate 102 and the associated cavities 104 are disposed on an underlying substrate 106. Similar to the previous embodiments, the supporting substrate 102 can be formed of any suitable material, such as cured sol-gel precursor materials. In some embodiments, the underlying substrate 106 is formed of glass, though other materials can also be employed. In some embodiments, the substrates 102 and 106 are substantially transparent to one or more wavelengths of radiation that can interact with metal coatings provided on the top surface of the substrate 102 and/or at least portions of the inner surfaces of the cavities (e.g., by exciting localized surface plasmons therein) so as to mediate the generation of pores in cells membranes, as discussed in more detail below.

With continued reference to FIG. 10, the cavities 104 are in the form of a vertical stack of interconnected cavities. In other words, there are multiple layers of cavities that are stacked on top of each other. The top layer of the cavities 104a includes openings 104b. Similar to the previous embodiments, a metal coating 108 covers a top surface of the substrate. Further, a portion of the inner surface of each of the cavities 104a is coated with a thin metal layer 110. Similar to the previous embodiments, the metal coatings 108 and 110 can be formed of the same or different materials. In some embodiments, gold or silver is employed for forming the metal coatings 108 and 110. In some embodiments, the coating 108 and/or 110 can be formed of TiN.

In some embodiments, the dimensions of the cavities can be similar to those discussed above in connection with the previous embodiments. At least one dimension of each cavity is in a range of about 50 nm to about 3.5 microns, e.g., in a range of about 100 nm to about 1 micron.

In some embodiments, a matrixsupporting a plurality of cavities arranged as vertically stacked layers can be formed employing the teachings of an article entitled “Assembly of large-area, highly ordered, crack-free inverse opal films,” authored by Hatton et. al. and published in PNAS (Jun. 8, 2010), which is herein incorporated by reference in its entirety. The structure can then be metalized, e.g., via vacuum metallization, so as to form a thin metal layer over the top surface of the substrate and a metal layer at the bottom of the inner surface of each cavity in the top-most layer of the vertically stacked cavity layers. [0077] A three-dimensional multilayer array of colloidal particles can be fabricated following a procedure described in the aforementioned Hatton et. al.'s article. For example, 1 mL of a 2.5 vol % aqueous colloidal suspension can first be added to 20 mL of distilled H₂O, in addition to up to 0.30 mL of hydrolyzed TEOS solution. The silica sol-gel solution can consist of tetraethylorthosilicate (TEOS), 0.1 M HCl and ethanol with mass ratios of 1:1:1.5 and can be stirred at room temperature for about 1 hour. A glass substrate can be cleaned in piranha solution and suspended in the colloid/TEOS suspension. The solvent can be evaporated in a 65° C. oven for 1-2 days, enabling the deposition of a thin film of colloids onto the glass substrate.

To form the inverse colloidal film, the substrate can then be baked at 500° C. for 2 hours, with a 4 hour ramp time. The baking process can remove the colloidal particles and can partially sinter the SiO₂ structure, leaving a 3D array of interconnected cavities on the glass substrate. A 2 nm film of titanium followed by a 3 0nm film of gold can then be evaporated onto the top layer of cavities to create metal-covered nanocavities.

In some embodiments, the 3D plasmonic structure 100 can be employed to cause perforation of cells membranes by placing a plurality of cells on the metal-coated top surface of the structure, and irradiating the structure so as to excite localized surface plasmons, which in turn can dissipate the applied radiation, e.g., via heat generation, thereby facilitating the formation of pores in the cells membranes. For example, similar to the previous embodiments, the dissipation of the applied electromagnetic energy can result in generation of bubbles in a medium in which the cells are disposed, where the bubbles cause poration of the cells membranes. The radiation can be applied to the structure from the above the top surface, or alternatively from below the structure, e.g., where the substrates 102 and 106 are transparent to the radiation. While in some embodiments, pulsed radiation is employed in others CW radiation is applied to the plasmonic structure 100. The parameters of the applied radiation can be similar to those discussed above in connection with the previous embodiments.

A 3D plasmonic structure according to the present teachings can provide a number of advantages. For example, it can allow molecules (e.g., dyes, DNA plasmids, etc.) to be actively delivered to perforated cells via the interconnected porous layers of the structure.

The following examples are provided to further elucidate various aspects of the invention. The examples are not intended to necessarily indicate the optimal ways of practicing the invention or the optimal results that can be obtained, but rather are provided for illustrative purposes.

### EXAMPLE 1

### Fabrication of a Plasmonic Substrate

An inverse colloidal monolayer was formed following a procedure described in the above mentioned article entitled “Transparency and damage tolerance of patternable omniphobic lubricated surfaces based on inverse colloidal monolayer” by Vogel et al. published in Nature Communications (2013). The colloids were fabricated by surfactant free emulsion polymerization (Vogel et al., “A Convenient Method to Produce Close and Non-Close-Packed Monolayers Using Direct Assembly At The Air-Water Interface And Subsequent Plasma-Induced Size Reduction,” Macromolecular Chemistry and Physics. 2011, 212, 719), (Jun. 20, 2011). First, filtered water was heated to 80° C. and degassed. Styrene or methyl methacrylate (MMA) monomer was stirred into the water phase, and an aqueous solution of dissolved sodium styrene sulfonate or acrylic acid comonomer was added to the mixture. A potassium peroxodisulfate (KPS) initiator was dissolved in water and added to the mixture, which was then heated to 80° C. to achieve polymerization. After cooling the mixture to room temperature, the mixture was purified by filtration and dialyzation.

To form a two-dimensional array of colloidal particles, an aqueous suspension of the synthesized colloids was diluted with ethanol to form a 1:1 water/ethanol colloidal dispersion. The colloidal dispersion was deposited at the air-water interface via a partially immersed hydrophilic glass slide tilted at an angle of approximately 45 degrees with respect to the water surface. A hydrophilic glass substrate was then immersed into the subphase and manually elevated to transfer the monolayer from the air-water interface to the surface of the glass substrate.

To form the inverse monolayer, a silica sol-gel solution of tetraethylorthosilicate (TEOS), 0.1 M HCl and ethanol with mass ratios of 1:1:1.5 was prepared and stirred for 1 hour. The solution was then diluted with ethanol at a 1:1 ratio and spin-coated onto the monolayer-covered glass substrate at 3000 rpm for 30 seconds to cover the monolayer to a height of approximately two-thirds the diameter. The substrate was then baked at 500° C. for 5 hours to remove the colloids, leaving an array of interconnected cavities on the glass substrate. A 2 nm film of titanium followed by a 30 nm film of gold was then evaporated onto the cavities to create a continuous film of gold-covered nanocavities.

The gold-covered nanocavity substrate was then attached to the bottom of a 35 mm glass-bottom petri dish with a 14 mm-diameter circular hole with UV-curable glue. FIG. 7 shows a top-view SEM image of the nanocavities.

To increase biocompatibility of the sample and promote adhesion of the cells to the substrate, the petri dish was soaked in cell growth media solution for approximately 2 hours prior to seeding of cells.

### Culturing Cells

Experiments were performed using Hela S3 cancer cells that were cultured in 75 cm² cell culture flasks with vented caps and placed in a humidified incubator with 5% CO₂ atmosphere at 37° C. Cells were passaged every 2-3 days when 80% confluency was reached. Growth media solution was prepared using Dulbecco's Modified Eagle medium (DMEM) with 10% fetal bovine serum and 1% penicillin-streptomycin.

For poration experiments, 0.3 x 10⁶ cells were seeded per 35-mm petri dish 24 hours prior to laser treatment.

### Porating Cells Using Femtosecond Laser System

Cell poration was achieved using a Ti:Sapphire laser emitting 100-fs pulses with an 800-nm central wavelength at a repetition rate of 250 kHz in a manner discussed above. The laser power was varied using a combination of a half-wave plate and a polarizing beam-splitter, and exposure time was controlled with a mechanical shutter. An illuminated axicon of opening angle 5° created a Bessel beam, which was focused onto the sample using a low numerical aperture objective (the numerical aperture was about 0.17). The sample was placed on a mechanical stage with three-dimensional motion control. The stage was used to scan the laser spot over the sample at a speed of 1000 μm/s in x and y directions. The fluence of the applied pulses was 10³J/cm².

Labeling Cells with Dyes

Immediately before treatment, 200 μL of 900 μM green fluorescent calcein dye solution was added to the petri dish. The green fluorescent calcein dye solution was prepared by dissolving 0.57 mg of powdered calcein green dye per mL of phosphate-buffered saline (PBS).

After treatment, cells were washed twice with PBS (phosphate-buffered saline). Cell growth media was added to the petri dish and the cells were incubated for 1 hour. The cells were then washed once with PBS and 2 mL of a calcein red-orange AM dye solution was added to the petri dish. The dye solution was prepared by dissolving 50 μg of calcein red-orange AM in dimethyl sulfoxide (DMSO) and diluting in PBS to a final concentration of 1 μg/mL. The cells were incubated for 10 minutes. To reduce background fluorescence, the dye solution was replaced with PBS before imaging.

### Assessing Cell Viability and Poration Efficiency

To quantify cell viability, cells were imaged using fluorescent microscopy 1 hour after treatment to detect a calcein red-orange AM signal. In viable cells, the non-fluorescent calcein red-orange AM dye molecules are converted to fluorescent calcein after ester hydrolysis by active intracellular esterases. Cell viability was calculated by dividing the total number of cells with a detectable calcein red-orange AM signal by the total number of cells treated.

To quantify poration efficiency, cells were imaged using fluorescent microscopy 1 hour after treatment to assess detection of a calcein green signal. Calcein green is membrane-impermeable and will only be taken up by porated cells. Poration efficiency was calculated by dividing the total number of cells with a detectable calcein green signal by the total number of cells treated.

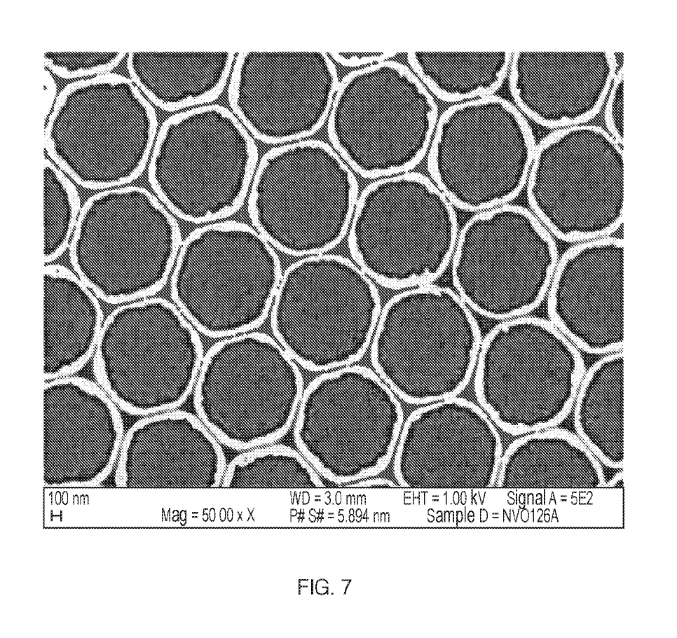

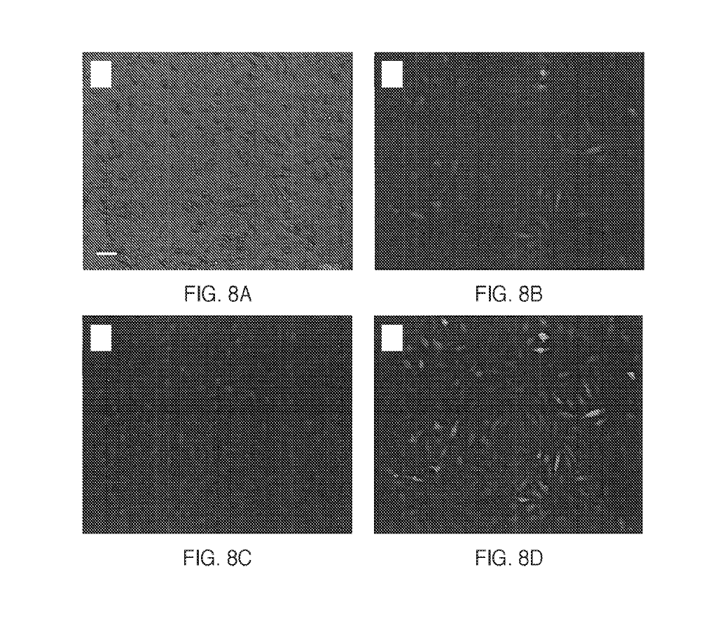

FIG. 8A shows that the HeLa cancer cells adhered to the plasmonic structure. FIG. 8B shows that the HeLa cells were successfully porated. Specifically, observation of green fluorescence indicates the uptake of the membrane-impermeable Calcein green dye by the porated cells. FIG. 8C shows that the porated cells remained viable after treatment as evidenced by the observation of blue fluorescence indicating the uptake of Calcein AM red by those cells and the breakage of Calcein AM ester bond via enzymatic activity of those cells. FIG. 8D is an overlay image indicating the porated and viable HeLa cells after treatment.

### EXAMPLE 2

Electromagnetic simulations were performed to understand the interaction of the incoming laser light with the plasmonic nanocavities according to the present teachings and to optimize the structure for strong field enhancement. The numerical software Comsol Multiphysics 4.3b, marketed by Comsol, Inc. of Burlington Mass., U.S.A. was employed for performing the simulations because it is suitable for simulating complex and curved geometries, and because it allows combining the electric field simulation with a temperature model for the structure.

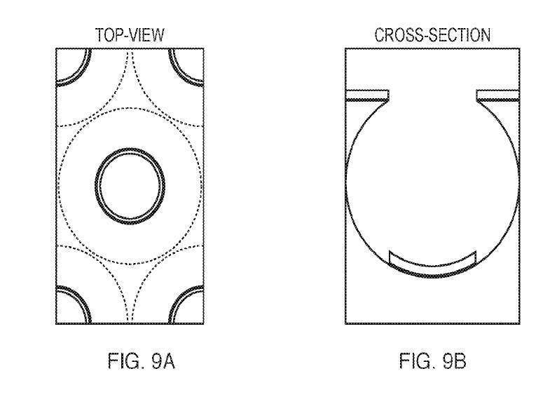

The conductive coating was assumed to be a gold coating, and the following parameters were utilized to characterize the structure: cavity radius (r), aperture size of the cavity (a), and the gold thickness (d). The structure itself was created with the Comsol Model builder. All edges were rounded with a curvature of 5 nm to avoid numerical artifacts. FIGS. 9A and 9B schematically show one of the cavities. The diameter of the cavity was selected to be 1 micrometer, the aperture diameter was 500 nanometers and the thickness of the gold film was 30 nanometers.

The optical properties of the gold were taken from an article by P. B. Johnson and R. W. Christy; Optical Constants of the Noble Metals; Phys. Rev. B 6, 4370-4379 (1972), which is herein incorporated by reference in its entirety.

The refractive index of the water and silica was set to 1.33 and 1.54, respectively. In areas of strong field enhancement, a maximal mesh size of 3 nm and everywhere else a maximum mesh size of 50 nm were employed. To exploit the symmetry of the system, periodic and antiperiodic boundary conditions were applied in the x and y directions. The simulation domain was truncated with perfectly matched layers in the z-direction. The incoming laser field was set up using the scattered field formulation discussed in Guillaume Demésy, Laurent Gallais, France Mireille Commandre; Tridimensional multiphysics model for the study of photo-induced thermal defects in arbitrary nano-structures; Journal of the European Optical Society—Rapid Publications 6, 11037 (2011), which is herein incorporated by reference in its entirety.

In order to characterize the strength of the field enhancement of different cavity structures, a Figure of Merit (FoM) was defined as the average local E-field enhancement on the edge of the aperture and on the edge of the disk. The choice of this FoM is supported by two arguments:
Evaluating the average instead of the maximum field enhancement is more robust as it averages out potential numerical artifacts in single data points, and
A contour average as observable is independent of the aperture size increase in contrast to an area average over the aperture

FIGS. 6A and 6B discussed above show the results of the simulations, showing that “hotspots” can be generated at the rim and the bottom of the cavities.

### EXAMPLE 3

In some embodiments, the metal-coated nanocavity structured matrices according to the present teachings can be used to deliver membrane-impermeable calcein and dextran dyes into cells using both nanosecond and femtosecond laser pulses. Near-field scanning optical microscopy images confirm the regions of near-field enhancement on the top rims of the nanocavities as suggested by the finite element method simulations. Pump-probe measurements indicate that bubbles form on the gold-coated nanocavity structured matrices at a laser fluence of approximately 30.8 mJ/cm², which is lower than the bubble threshold of 130 mJ/cm² for a flat gold substrate.

### Culturing Cells

The following experiments were performed using Hela CCL2 cervical cancer cells.

### Porating Cells Using Nanosecond Laser Radiation

Cell poration was achieved using two separate nanosecond laser systems: an Nd:YAG laser emitting 4-ns pulses with a 1064 nm central wavelength at a repetition rate of 10 Hz, and an Nd:YAG laser emitting 11-ns pulses with a 1064 nm central wavelength at a repetition rate of 50 Hz. Similar to the previous examples, the cells were disposed on a plasmonic nanocavity-structured matrix according to the present teachings. which included cavities that were 1 micron deep with an aperture of approximately 350 nm in diameter. The metal coating applied to the top surface of the matrix and the inner surface of the nanocavities was formed by a 2-nm of an adhesive titanium layer underlying a 50-nm film of gold. The laser power was varied using a combination of a half-wave plate and a polarizing beam-splitter, and exposure time was controlled with a mechanical shutter. In some experiments a Gaussian beam was focused onto the sample using a low numerical aperture objective (numerical aperture of approximately 0.17) and in some experiments a top-hat beam was focused onto the sample using a low numerical aperture objective (numerical aperture of approximately 0.17). The sample was placed on a mechanical stage with three-dimensional motion control. The stage was used to scan the laser spot over the sample at a speed of 1000 μm/s in x and y directions. The fluence of the applied pulses was on the order of 10⁻³ J/cm².

Labelling Cells with Dyes

Membrane-impermeable calcein dye and membrane-impermeable fluorescein isothiocyanate dextran dye were both successfully delivered to porated cells. For the calcein delivery, immediately before treatment, 200 μL of 900 μM green fluorescent calcein dye solution was added to a petri dish containing 2 mL of warm cell media. The green fluorescent calcein dye solution was prepared by dissolving 0.57 mg of powdered calcein green dye per mL of phosphate-buffered saline (PBS). For the dextran delivery, immediately before treatment, the cell media was replaced by 1 mL of warm PBS containing 25 mg of dissolved dextran dye.

### Assessing Cell Viability and Poration Efficiency

### Method

To quantify poration efficiency, cells were imaged using fluorescent microscopy 1 hour after treatment to assess detection of a calcein green or dextran signal. Calcein green and dextran are both membrane-impermeable and will only be taken up by porated cells. Poration efficiency was calculated by dividing the total number of cells with a detectable calcein green or dextran signal by the total number of cells treated. The viability of radiation-treated cells was assessed by determining whether they provide their normal enzymatic activity. In particular, the treated cells were exposed to Calcein-AM, which is membrane permeable and hence is uptaken by all cells. Calcein-AM is not, however, naturally fluorescent. It fluoresces only when its ester bond is broken by an enzyme within the cell. Thus, if a treated cell that has uptaken Calcein-AM exhibits a fluorescent signal associated with Calcein-AM, then one can conclude that it has maintained its healthy enzymatic activity and will be herein considered as a viable cell.

### Results

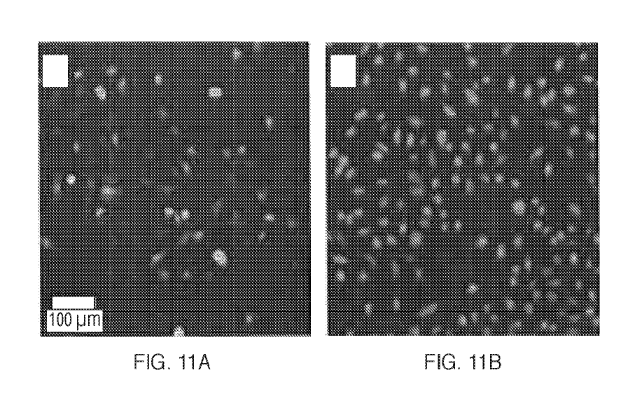

FIG. 11A is a fluorescent image showing that upon illumination with a laser emitting 4-ns pulses at a repetition rate of 10 Hz with a central wavelength of 1064 nm, approximately 46% of the HeLa cells uptake calcein. FIG. 11B is another fluorescence image showing that approximately 99% of the radiation-treated cells remain viable, as evidenced by their ability to uptake Calcein-AM and enzymatically break its ester bond.

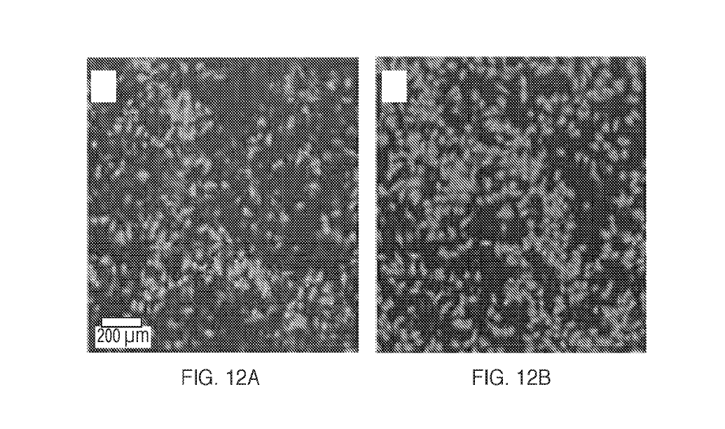

FIG. 12A is a fluorescence image showing that upon illumination with a laser emitting 11-ns pulses at a repetition rate of 50 Hz with a central wavelength of 1064 nm, approximately 34% of the HeLa cells uptake dextran. FIG. 12B shows that approximately 81% of the radiation-treated cells were viable, as evidenced by their ability to uptake Calcein-AM and enzymatically break its ester bond.

### Porating Cells Using Femtosecond Laser Radiation

The experiment discussed above in connection with nanosecond pulses were also performed using femtosecond pulses. The results were as follows.

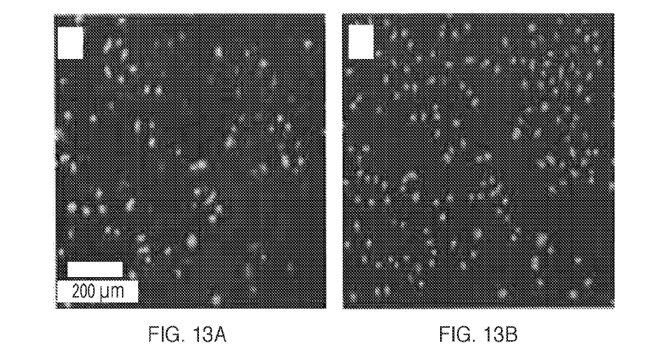

FIG. 13A is a fluorescent image showing that upon illumination with a laser emitting 100 fs pulses at a repetition rate of 10 kHz with a central wavelength of 800 nm, approximately 84% of HeLa cells uptake calcein. FIG. 13B is a fluorescent image showing that approximately 92% of the radiation-treated cells remained viable, as evidenced by their ability to uptake Calcein-AM and enzymatically break its ester bond.

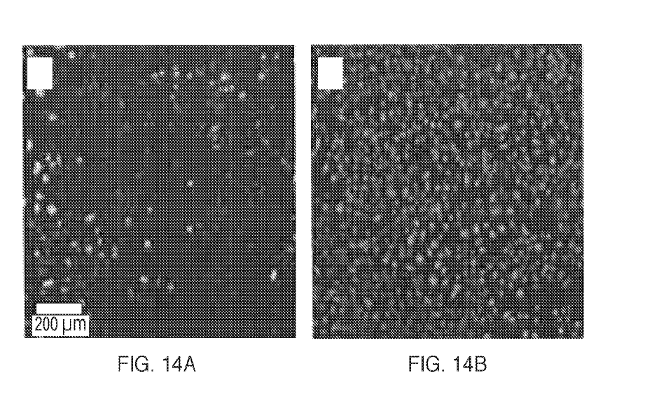

FIG. 14A is a fluorescent image showing that upon illumination with a laser emitting 100 fs pulses at a repetition rate of 10 kHz with a central wavelength of 800 nm, approximately 25% of the HeLa cells uptake dextran. FIG. 14B is a fluorescent image showing that approximately 79% of the radiation-treated cells remained viable, as evidenced by their ability to uptake Calcein-AM and enzymatically break its ester bond.

Those having ordinary skill in the art will appreciate that various changes can be made to the above embodiments without departing from the scope of the invention. All of the aforementioned publications are herein incorporated by reference in their entirety.

1. A plasmonic structure for use in transfecting cells, comprising:
a matrix supporting a plurality of cavities, each cavity having an opening and an inner surface extending beneath said opening,
a layer of an electrically conductive material coating at least a portion of said matrix ,wherein at least one dimension of each of said cavities is in a range of about 50 nm to about 2 microns.

2. The plasmonic structure of claim 1, wherein said electrically conductive material coats at least a portion of a top surface of said matrix.

3. The plasmonic structure of claim 1, wherein said electrically conductive material coats at least a portion of an inner surface of at least one of said cavities.

4. The plasmonic structure of claim 1, wherein said electrically conductive material coats at least a portion of a top surface of said matrix and at least a portion of an inner surface of at least one of said cavities.

5. The plasmonic structure of claim 1, wherein said electrically conductive coating coats an entire top surface of said matrix and an entire inner surface of each of said cavities.

6. The plasmonic structure of claim 1, wherein said at least one dimension is in a range of about 100 nm to about 1 micron.

7. The plasmonic structure of claim 1, wherein said at least one dimension is in a range of about 200 nm to about 800 nm.

8. The plasmonic structure of claim 1, wherein all dimensions of each of said cavities are in a range of about 100 nm to about 1 micron.

9. The plasmonic structure of claim 1, wherein said at least one dimension corresponds to a diameter of said opening of each of said cavities.

10. The plasmonic structure of claim 1, wherein said at least one dimension corresponds to depth of each of said cavities.

11. The plasmonic structure of claim 1, wherein said electrically conductive layer has a thickness in a range of about 10 nm to about 100 nm.

12. The plasmonic structure of claim 1, wherein said layer of an electrically conductive material comprises TiN.

13. The plasmonic structure of claim 1, wherein said layer of an electrically conductive material comprises a metal.

14. The plasmonic structure of claim 13, wherein said metal is any of gold and silver.

15. The plasmonic structure of claim 1, wherein said matrix is formed of a biocompatible material.

16. The plasmonic structure of claim 1, wherein said cavities have truncated spherical shapes.

17. The plasmonic structure of claim 1, wherein said cavities are configured to permit placement of at least one cell over said cavities such that the cell extends over a plurality of openings of said cavities.

18. The plasmonic structure of claim 1, wherein said layer of an electrically conductive material is configured to generate localized surface plasmons in response to irradiation thereof by a plurality of short laser radiation pulses.

19. The plasmonic structure of claim 14, wherein said localized surface plasmons dissipate the incident radiation energy to generate hot spots at or in proximity of the rims of said cavities over which the cell is disposed such that said hot spots mediate poration of said cell.

20. The plasmonic structure of claim 18, wherein said matrix is substantially transparent to said radiation pulses.

21. The plasmonic structure of claim 18, wherein said laser pulses have a pulse duration in a range of about 100 femtoseconds to about 1 picosecond.

22. The plasmonic structure of claim 18, wherein said laser pulses have a pulse duration in a range of about 100 femtoseconds to about 50 nanoseconds.

23. The plasmonic structure of claim 1, wherein said plurality of cavities comprise vertically stacked layers of a plurality of interconnected cavities.

24. A method of causing poration of cells, comprising:
placing at least one cell over a top surface of a matrix supporting a plurality of cavities, wherein at least a portion of said matrix is coated with an electrically conductive layer and wherein at least one dimension of each of said cavities is in a range of about 50 nm to about 2 microns,
irradiating said matrix with a laser radiation such that an interaction of said laser radiation with said electrically conductive layer mediates the generation of one or more pores in the membrane of said at least one cell.

25. The method of claim 24, wherein said laser radiation comprises a plurality of laser pulses.

26. The method of claim 25, wherein said laser pulses generate localized surface plasmons in the electrically conductive layer, wherein said localized surface plasmons dissipate the laser radiation, thereby effecting formation of pores in the membrane of said at least one cell.

27. The method of claim 24, wherein said at least one cell extends at least partially over a plurality of said cavities.

28. The method of claim 25, wherein said laser pulses have a pulse duration in a range of about 10 femtoseconds to about 1 picosecond.

29. The method of claim 25, wherein said laser pulses have a pulse duration in a range of about 100 femtoseconds to about 100 nanoseconds.

30. The method of claim 29, wherein said laser pulses have a pulse duration in a range of about 1 nanosecond to about 100 nanoseconds.

31. The method of claim 26, wherein said at least one cell is disposed in a medium and wherein said localized surface plasmons lead to generation of heat, thereby generating bubbles in said medium such that said bubbles effect formation of pores in the cell's membrane.

32. The method of claim 25, wherein said laser pulses have a fluence in a range of about 1 mJ/cm² to about 100 mJ/cm² at said matrix.

33. The method of claim 24, wherein said radiation irradiates the matrix from below said cavities.
